(19) United States
(12) Patent Application Publication
(10) Pub. No.: US 2025/0315841 A1
(43) Pub. Date: Oct. 9, 2025

(54) SYSTEM FOR MEASUREMENT OF GREENHOUSE GASES AND AUTOMATIC GENERATION OF CARBON CREDITS IN FOREST AREAS

(71) Applicant: SOUTH BRAZIL MOBILITY LTDA, Porto Alegre (BR)

(72) Inventors: Alexandre De Souza Tavares, Porto Alegre (BR); Arnildo Schildt, Caxias do Sul (BR)

(21) Appl. No.: 18/886,392

(22) Filed: Sep. 16, 2024

(30) Foreign Application Priority Data
Apr. 8, 2024 (BR) BR102024006823-8

(51) Int. Cl. G06Q 30/018; G01N 33/00

(52) U.S. Cl. CPC G06Q 30/018; G01N 33/0036; G01N 33/0075

(57) ABSTRACT

A system for measuring greenhouse gases and automatic generation of carbon credits in forest areas, based on real-time data, which allows online analysis, generating automatic certification of carbon credits. The system presents a microprocessor unit (UP) that receives signals from sensors measurement of gases (Sg) analyzed in a chromatography unit High Efficiency Liquid (HPLC) and rainfall sensors (Sp). The data is processed in the local processing unit (UP) and transmitted to a remote processing unit (UPr) through the communication module (MC), using transmission protocols of data. The processed data generates credit certificates for authenticated and authorized by the UNFCCC (Convention-United Nations Framework on Climate Change), ensuring its internationally recognized validity.

### CROSS REFERENCE TO RELATED APPLICATION

This application claims the benefit of and takes priority from Brazilian patent application Ser. No. 10/202,4006823-8 filed on Apr. 8, 2024, the contents of which are herein incorporated by reference.

### FIELD OF INVENTION

The present invention describes a system for the measurement of greenhouse gases and the automatic generation of greenhouse gas carbon credits in forest areas, based on real-time data, which allows online analysis, generating automatic certification of carbon credits.

### BACKGROUND OF THE INVENTION

The global trend in greenhouse gas (GHG) emissions has increased since the beginning of the 21st century compared to the three previous decades, mainly due to the increase in emissions of CO2 from emerging economies. As a result, the concentration of greenhouse gases in the atmosphere has increased substantially, negatively affecting life on Earth.

Under the Kyoto Protocol (1997), seven greenhouse gases greenhouses are considered to contribute to global warming: Carbon dioxide (CO2), Methane (CH4), Nitrous oxide (N2O), Hydrofluorocarbons (HFCs), Perfluorocarbons (PFCs), Sulfur hexafluoride (SF6), and Nitrogen trifluoride (NF3)3. The CO2 emissions from fossil fuels are the largest source of global emissions, especially due to the burning of fossil fuel for energy generation.

In 2015, the Paris Agreement determined parties' efforts to reduce greenhouse gas emissions include (1) establishing a specific emission limit GHG that a company cannot exceed, (2) introducing a tax on carbon where the company pays for the amount of CO2 it produces, and (3) implementing an emissions trading scheme to create a carbon market.

The last solution has gained momentum recently due to the positive incentive from clean energy producers, encouraging the fossil fuel industry to become more efficient and gradually reduce its emissions. This led to the creation of carbon credits. A carbon credit is a tradable license or certificate which gives the right to emit one ton of carbon dioxide or equivalent of another greenhouse gas.

There are currently two types of carbon credits: emissions voluntary reduction, which comprises compensation of carbon exchanged on the over-the-counter and voluntary market for credits and certified reduction of emissions, where emission units (or credits) are created through a regulatory framework with the objective to offset a project's emissions. The latter requires a certifying body for regulation.

The prior art describes systems and methods for quantifying GHG emissions from industrial and urban activities.

Document WO2023168524 describes a system and a method for quantifying greenhouse gas emissions (GHG) associated with automobile traffic. The method includes receiving data from a plurality of devices located in a plurality of traffic intersections in a traffic network, each device comprising at least one sensor configured to obtain corresponding traffic intersection data. The data received are used in conjunction with an emissions model to obtain the corresponding data on greenhouse gas emissions greenhouse effect. A methodology for quantifying effect gases greenhouse GHG, based on ISO 14064-2:2019, determines a reduction that can achieve a carbon offset credit.

Document CN110807175 describes a measurement method and calculation of carbon emissions based on model data target urban traffic. According to the invention, model data local traffic data from a target city are used as a basis for establish a method for measuring and calculating carbon emissions more accurately.

Document WO2024026248 describes an ecosystem of climate technology that automates the collection and processing of data through computer software programmed to generate automatically encrypted carbon credit certificates uniforms that can be traded on peer-to-peer exchanges or as derivatives. The ecosystem creates a network of information derived from nine systems designed to: sensor, qualify, measure, code, encrypt, issue, index, record, and exchange, all of which depend of artificial intelligence to identify, calculate, and quantify the source and carbon origin and automatically generate carbon credit certificates uniform encrypted carbons based on the nature and attributes of the carbon and associated CO2e.

Likewise, the state of the art describes systems and methods that quantify GHG emissions and convert them into carbon credits.

Document WO2023229540 describes a system and method for real-time digital monitoring to verify greenhouse gas emissions and the conversion to digital assets. The method comprises a step for monitoring emissions of greenhouse gases, which is the stage of acquiring data from sources of data and sending data to the blockchain network to validate and verify the transaction by proof of history of the blockchain network, a stage of processing raw data into primary data raw data on the blockchain network that meets ISO 14064, a step to check greenhouse gas emissions on the blockchain network, configuring a smart contract validation node. The result is recorded on the blockchain network and can be viewed through applications and dash boards via Web 3.0 technology. The result, which is the credit of carbon, can be converted into a digital asset in the form of a digital token.

The prior art describes systems and methods for quantification of emissions and conversion into carbon credits industrial and urban activities. However, no systems exist for accounting for the amount of carbon dioxide emissions in forest area.

In this way, the Applicant developed a system that meets rigorous measurement standards, based on data acquisition through sensors and data obtained from external sources, such as rainfall maps, carrying out analysis of greenhouse gases in time real, with online analysis, generating automatic certification of carbon credits.

### SUMMARY

The invention describes a system for measuring greenhouse gases and the automatic generation of carbon credits based on real-time data that provides accurate measurement of air emissions greenhouse gases (GHG), certification and automatic generation of carbon credits.

The invention describes a system for measuring greenhouse gases that reduces the time for analyzing and measuring carbon credits (currently between 24 and 30 months) for an online analysis, generating automatic certification of carbon credits.

### BRIEF DESCRIPTION OF FIGURES

FIG. 1 presents the schematic representation of the system for measuring greenhouse gases and automatically generating carbon credits in forest areas.

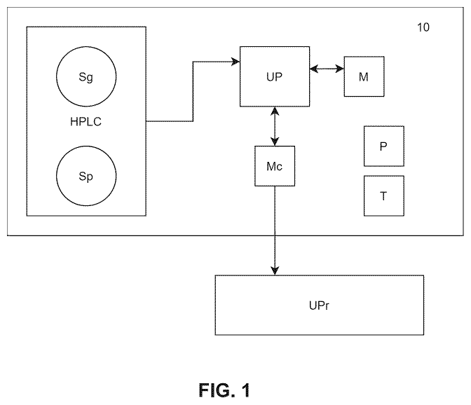

### DETAILED DESCRIPTION OF THE INVENTION

The system for measuring greenhouse gases and generating automatic carbon credits in forest areas, object of the present invention, comprises a cabinet made of resistant material (10), equipped with a control panel (P) and a screen (T) for data presentation.

A local processing unit (UP) is arranged in the cabinet (10). The local processing unit (UP) is equipped with memory (M), a communication module (MC) that receives and sends data to remote units and a source of food.

In memory (M), data obtained from sources is stored as external data, such as atmospheric maps.

A High Performance Liquid Chromatography (HPLC) unit analyzes the collected gases and identifies the sample gases through of gas measurement sensors (Sg).

Gas measurement sensors (Sg) can be selected among Carbon Dioxide (CO2) measurement sensors; Methane (CH4); Nitrous Oxide (N20); Ozone (03); HFC 23 (CHF3); HFC-32 (CH2F2); HFC-41 (CH3F); HFC-43-10mee; (C5H2F10); HFC-125 (C2HF5); HFC-134(C2H2F4); HFC-134a (CH2FCF3); HFC-152^{a} (C2H4F2); HFC-143 (C2H3F3); HFC-143a (C2H3F3); HFC-227ea (C3HF7); HFC-236fa (C3H2F6); HFC 145ca (C3H3F5); hexafluoride Sulfur (SF6); Perfluoromethane (CF4); Perfluoroethane (C2F6); Perfluoropropane (C3F8); Perfluorocyclobutane (c-C4F8); Perfluoropentane (C5F12); Perfluorohexane (C6F14); (NF3)3 Nitrogen Trifluoride.

Rainfall sensors (Sp) obtain precipitation data rainfall, which are recorded and sent to the local processing (UP).

Data measured by gas sensors (Sg) and rain gauges (Sp) are forwarded to the local processor (UP) that performs the instructions of a computer program, using metrics specific for quantifying greenhouse gas emissions stove.

The power source may include rechargeable batteries for electrical energy or alternative energy, being particularly useful in remote access locations without electrical grid infrastructure.

Pre-processed data in the processing unit (UP) are transmitted to a remote processing unit (UPr) through the communication module (MC), using communication protocols data transmission.

From the processed data, certificates of carbon credits are authenticated and authorized by the UNFCCC (United Nations Framework Convention on Climate Change), ensuring their internationally recognized validity.

What is claimed is:

1. SYSTEM FOR MEASUREMENT OF GREENHOUSE GASES AND AUTOMATIC GENERATION OF CARBON CREDITS IN FOREST AREAS comprising a material cabinet resistant (10), equipped with a control panel (P) and a screen (T), featuring a local processing unit (UP), equipped memory (M), a communication module (MC) that receives and sends data to remote units and a power supply, characterized by presenting:
a High Performance Liquid Chromatography (HPLC) unit for analysis of the collected gas sample;
gas measurement sensors (Sg) that send signals to the unit local processing (UP);
Rainfall sensors (Sp) that send signals to the local processing (UP);
a Local processing unit (UP) with instructions from a computer program using specific metrics for the quantification of greenhouse gas emissions; and
a Remote processing unit (UPr) that receives data from the local processing unit (UP) through the communication (MC), using communication transmission protocols data.

2. SYSTEM FOR MEASUREMENT OF GREENHOUSE GASES AND AUTOMATIC GENERATION OF CARBON CREDITS IN FOREST AREAS, according to claim 1, characterized by gas measurement sensors (Sg) being selected from Carbon Dioxide measurement sensors (CO2); Methane (CH4); Nitrous Oxide (N20); Ozone (03); HFC 23 (CHF3); HFC-32 (CH2F2); HFC-41 (CH3F); HFC-43-10mee; (C5H2F10); HFC-125 (C2HF5); HFC-134 (C2H2F4); HFC-134a (CH2FCF3); HFC-152a (C2H4F2); HFC-143 (C2H3F3); HFC-143a (C2H3F3); HFC-227ca (C3HF7); HFC-236fa (C3H2F6); HFC 145ca (C3H3F5); Sulfur Hexafluoride (SF6); Perfluoromethane (CF4); Perfluoroethane (C2F6); Perfluoropropane (C3F8); Perfluorocyclobutane (c-C4F8); Perfluoropentane (CSF12); Perfluorohexane (C6F14); (NF3)3 Nitrogen Trifluoride.
